(19) United States
(12) Patent Application Publication
(10) Pub. No.: US 2026/0308682 A1
(43) Pub. Date: Oct. 8, 2026

(54) MICRORNA AS A DIAGNOSTIC TOOL AND FOR DEVELOPING ADVANCED THERAPIES OF AMYOTROPHIC LATERAL SCLEROSIS (ALS) WITH BULBAR ONSET

(71) Applicants: FONDAZIONE IRCCS ISTITUTO NEUROLOGICO CARLO BESTA, MILANO (IT); CONSIGLIO NAZIONALE DELLE RICERCHE, ROMA (IT)

(72) Inventors: Stefania MARCUZZO, Milano (IT); Erika SALVI, Milano (IT); Eleonora DALLA BELLA, Milano (IT); Davide PERICO, Roma (IT); Antonella DE PALMA, Roma (IT); Pierluigi MAURI, Roma (IT); Giuseppe LAURIA PINTER, Roma (IT)

(21) Appl. No.: 19/483,003

(22) PCT Filed: May 10, 2024
(86) PCT No.: PCT/IB2024/054563

§ 371 Date: Nov. 10, 2025

(30) Foreign Application Priority Data
May 11, 2023 (IT) IT202300009462

(51) Int. Cl. C12Q 1/6883; A61K 31/155

(52) U.S. Cl. CPC C12Q 1/6883; A61K 31/155; C12Q 2600/158; C12Q 2600/178

(57) ABSTRACT

The present patent application describes microRNAs for the diagnosis and development of advanced therapies for amyotrophic lateral sclerosis (ALS) with bulbar onset.

Amyotrophic lateral sclerosis (ALS) is a neurodegenerative disease which affects cortical, brainstem and spinal cord motor neurons, leading to skeletal muscle paralysis and cognitive-behavioral impairment. In the last three decades since the study which introduced riluzole as the first and, so far, only drug modifying the disease, albeit with poor clinically significant efficacy, all the subsequent studies have been designed using the clinical diagnosis of ALS as an eligibility criterion for patient enrollment.

This reflected the hypothesis that any subtype of disease, whether familial, sporadic, rapidly or slowly progressive, could share the same pathophysiology and potential response to the same drug. It was probably one of the main reasons for the failure of all phase 3 studies carried out to date.

ALS is a clinically heterogeneous syndrome the most urgent need of which is the achievement of a biological stratification of patients through specific signatures ranging from an aggressive to a very slow course, probably explained by several as yet unknown biological mechanisms. In clinical trials, the clinical heterogeneity is commonly resolved by dichotomization into bulbar and spinal forms.

Bulbar onset, which accounts for about 30% of cases, is the most homogeneous ALS phenotype, both in terms of progression and neuropathological features, and has guided the therapeutic effect of riluzole and, more recently, of guanabenz (Dalla Bella, E., et al. Brain 144, 2635-2647; 2021).

The bulbar onset phenotype is the most devastating variant of ALS, with a rapid decline in swallowing, language, and shorter survival.

Its clinical course is characterized by the early involvement of the upper motor neuron and cognitive-behavioral aspects.

Despite this, bulbar ALS is relatively understudied and basic research data are scarce due to limited accessibility to biological samples and lack of preclinical models summarizing the human phenotype.

Whatever the classification approach, bulbar onset is recognized as a unique phenotype.

The publication by Benigni Michele et al. (“Identification of miRNAs as Potential Biomarkers in Cerebrospinal Fluid from Amyotrophic Lateral Sclerosis Patients,” NEUROMOLECULAR MEDICINE, HUMANA PRESS, US, vol. 18, no. 4, 27 Apr. 2016, pages 551-560) describes the use of miR-150-5p as a diagnostic biomarker for ALS, but not for the specific diagnosis of the form of bulbar onset ALS.

The publication by Mignzhu Liu et al. (“The IGF2 intronic mi-483 selectively enhances transcription from IGF2 fetal promoters and enhances tumorigenesis”, GENES AND DEVELOPMENT, vol. 27, no. 23, 1 Dec. 2013, pages 2543-8 2543-8) describes the use of miR-483-5p as a diagnostic biomarker for ALS, but not for the specific diagnosis of the form of bulbar onset ALS.

The publication by Liguori Maria et al. (“Dysregulation of MicroRNA and Target Genes Networks in Peripheral Blood of Patients With Sporadic Amyotrophic Lateral Sclerosis,” FRONTIERS IN MOLECULAR NEUROSCIENCE, vol. 11, 28 Aug. 2018) describes miR-185-5p to distinguish the bulbar onset form of ALS.

### SUMMARY OF THE INVENTION

The inventors of the present patent application have surprisingly found that specific microRNAs are indicators of the form of ALS with bulbar onset; therefore, these can be used as an advanced diagnostic tool or for developing advanced therapies for this specific disease subtype.

### OBJECT OF THE INVENTION

In a first object of the invention, the use of specific microRNAs in the diagnosis of amyotrophic lateral sclerosis (ALS) with bulbar onset is described.

In a second object of the invention, a diagnostic method for the diagnosis of amyotrophic lateral sclerosis (ALS) with bulbar onset is described.

In a third object of the invention, the use of the same microRNAs as potential targets for developing new therapeutic approaches for amyotrophic lateral sclerosis (ALS) with bulbar onset is described.

In an aspect, the specific microRNAs described can be employed to identify compounds for the specific therapy of amyotrophic lateral sclerosis (ALS) with bulbar onset.

In a fourth object of the invention, a specific microRNA is described for medical use in evaluating the response to the treatment of patients affected by amyotrophic lateral sclerosis (ALS) with bulbar onset with guanabenz or with drugs acting on the endoplasmic reticulum stress and the cellular response defined as unfolded protein response.

### BRIEF DESCRIPTION OF THE DRAWINGS

FIG. 1 shows the values of miR-150-5p and miR-483-5p down-regulated in the serum of patients affected by bulbar onset ALS.

FIG. 2 shows the “Receiver Operating Characteristic” (ROC) used to evaluate the sensitivity and specificity of miR-150-5p and miR-483-5p as a biomarker which discriminates the subgroup of patients affected by bulbar onset ALS.

FIG. 3 shows the results obtained from proteomic analyses performed in the serum of patients affected by bulbar and spinal onset ALS and respective age- and sex-associated healthy controls. The principal component analyses (PCA) and the volcano plot show how the expression levels of the identified proteins are specifically associated with the bulbar, spinal and healthy control phenotype.

FIG. 4 shows a down-regulation of the miR-106b-5p expression levels in the serum of patients affected by bulbar onset ALS after treatment with guanabenz 32 mg/64 mg with respect to patients treated with placebo/16 mg. Interestingly, the expression levels of such microRNA tend to normalize and have expression values similar to those observed in the serum of healthy controls. Such a phenomenon has not been demonstrated in patients affected by spinal onset ALS, as shown in FIG. 4, in which the expression levels of such a molecule remain up-regulated, even after treatment with respect to the healthy controls.

### DETAILED DESCRIPTION OF THE INVENTION

In a first object of the invention, the use of specific microRNAs in the diagnosis of amyotrophic lateral sclerosis (ALS) with bulbar onset is described.

In particular, said microRNAs are:

| SEQ. ID. no. 1 | |
|---|---|
| miR-150-5p | UCUCCCAACCCUUGUACCAGUG |
| SEQ. ID. no. 2 | |
| miR-483-5p | AAGACGGGAGGAAAGAAGGGAG |

In particular, the down-regulation of miR-150-5p and miR-483-5p is specifically related to the phenotype of amyotrophic lateral sclerosis (ALS) with bulbar onset, determining a biological classification of such a phenotype.

For the purposes of the present invention, the down-regulation indicative of diagnosis is of one or both of the microRNAs described above, i.e., simultaneously.

Therefore, according to a second object, a diagnostic method for the diagnosis of amyotrophic lateral sclerosis (ALS) with bulbar onset in a patient suspected of having this specific form of disease is described.

More in particular, such a method comprises the step of determining the expression levels of the specific miR-150-5p and/or miR-483-5p microRNAs in a biological sample isolated from said patient suspected of having said disease.

More in particular, said biological sample isolated from said patient suspected of having said disease is represented by an isolated sample of serum from said patient.

The expression levels can be determined by techniques known in the art; in particular, it can be determined by real time PCR (rt-PCR).

The diagnosis is confirmed when the expression of said microRNAs is down-regulated.

For example, the following average results were obtained:

| | | spinal | | bulbar | bulbar | spinal |
|---|---|---|---|---|---|---|
| | | average | | vs spinal | vs HC | vs HC |
| | | Log2 | | Log2 | Log2 | Log2 |
| | | Fold | | Fold | Fold | Fold |
| | bulbar | Change | average | Change | Change | Change |
| | average | (FC) | HC | (FC) | (FC) | (FC) |
| miR-150-5p | 1.03 | −0.15 | −0.10 | −2.27 | −2.18 | −0.96 |
| miR-483-5p | 6.70 | 5.06 | 4.34 | −3.12 | −5.15 | −1.65 |

In a third object of the invention, the use of specific microRNAs as potential targets for developing new therapeutic approaches for amyotrophic lateral sclerosis (ALS) with bulbar onset is described.

In particular, said microRNAs are represented by: miR-150-5p and miR-483-5p.

The modulation of the expression of the specific microRNAs: miR-150-5p and miR-483-5p towards physiological levels allows the evaluation and development of new therapies for amyotrophic lateral sclerosis (ALS) with bulbar onset.

In fact, such therapies are effective when they have the effect of down-regulating the expression of the two specific miRNAs, either independently of each other or both.

In a fourth object of the invention, a specific microRNA is described for medical use in the treatment of patients affected by amyotrophic lateral sclerosis (ALS) with bulbar onset with guanabenz or drugs acting on the endoplasmic reticulum stress and the cellular response defined as unfolded protein response.

In particular, said medical use is in evaluating the response to treatment with guanabenz of patients affected by amyotrophic lateral sclerosis (ALS) with bulbar onset.

More in particular, the treatment with guanabenz comprises the administration to a patient in need of guanabenz 32 mg or 64 mg (daily dose).

For the purposes of the present invention, said specific microRNA is represented bv miR-106b-50:

| SEQ. ID. no. 3 | |
|---|---|
| miR-106b-5p | UAAAGUGCUGACAGUGCAGAU |

In particular, the medical use of the miR-106b-5p microRNA comprises the evaluation of the expression levels of said microRNA in which isolated sample of serum of the patient during the treatment with guanabenz.

The expression value of said miRNA will provide indications about the effects of the drug treatment.

In fact, it was surprisingly found that after treatment with guanabenz with a dosage of 32 mg or 64 mg (daily dose), the expression levels of miR-106b-5p are down-regulated in the serum of patients with amyotrophic lateral sclerosis (ALS) with bulbar onset.

Such expression levels, after treatment, show a trend similar to that observed in the serum of healthy controls.

### Experimental Part

To discover a distinct molecular signature of the bulbar onset of ALS, an integrated serum microRNA and proteomic profile was conducted. In a first exploratory phase, a cohort of 26 ALS patients and 28 healthy controls (HC) the serum samples of whom were analyzed by microfluidic techniques with TaqMan Human MicroRNA arrays containing 754 miRNAs was included in the study. The validation of the expression of the circulating microRNAs selected in the exploratory phase was carried out in a second independent cohort of 28 ALS and 22 HC patients by real-time PCR using specific Taqman assays. The proteomic analysis was performed in the serum of a subset of 14 ALS patients and 6 HCs included in the selected miRNA validation study. The integration of the molecular data obtained from the analysis of miRNA expression levels and proteomic data revealed a distinct profile of patients affected by bulbar onset ALS.

### Demographic and Clinical Features.

The study included a total of 54 ALS patients and 50 HCs who met the diagnostic criteria for ALS recruited from the Motor Neuron Disease Center of the IRCCS Fondazione Istituto Neurologico Carlo Besta in Milan, Italy, as part of the TRANS-ALS projects (concession no. 2015-0023 to G.L.) and INTERSLA (concession no. 1157625 to G.L.). The ALS and HC patients did not differ significantly by sex (p=0.67 exploration phase, p=0.85 validation phase) and age (p=0.74 exploration phase, p=0.62 validation phase). Bulbar onset was diagnosed based on the presence of dysarthria or dysphagia, tongue atrophy and fasciculation without spinal involvement of the lower motor neuron in the first 6 months after the presentation of symptoms and with present or subsequent signs of the upper motor neuron. Pseudo-bulbar onset defined as prominent bulbar features due to upper motor neuron involvement were excluded from the transcranial corticobulbar magnetic stimulation in all patients.

Exploration Phase-Identification of Dysregulated microRNAs in the Serum of Patients Affected by Bulbar Onset ALS.

An expression profile of 754 miRNAs was performed, by microfluidic techniques, in serum samples from 26 ALS and 28 HC patients to identify relevant molecules potentially implicated in the bulbar onset phenotype. Specifically, the microRNAs differentially expressed in patients affected by bulbar onset ALS (7), spinal onset (19), spinal onset and bulbar impairment due to ALS progression (16) and spinal onset without bulbar impairment (10) were considered. The exploration phase revealed four microRNAs (miR-885-5p, miR-150-5p, miR-483-5p, miR-342-3p) which were significantly down-regulated in the serum of patients with bulbar onset with respect to spinal onset ALS patients and HCs (Table 1).

TABLE 1

| | Bulbar | Bulbar vs | Spinal | Average | Average | Average | FC Bulbar | FC Bulbar |
|---|---|---|---|---|---|---|---|---|
| | onset | spinal | onset | expressed in | expression in | expression in | vs spinal | onset vs |
| MilNumberID | vs HC | onset | vs HC | bulbar onset | spinal onset | HCs | onset | HC |
| Hsa-miR-885-5p | 0.034* | 0.004* * | 0.13 | 3.98 ± 0.8 | 1.90 ± 1.56 | 2.67 ± 1.43 | −4.23 | −2.49 |
| ID 002296 | | | | | | | | |
| Hsa-miR-15O-5p | 0.004* | 0.009* | 0.61 | 1.69 ± 0.43 | 0.79 ± 0.92 | 0.60 ± 0.79 | −1.87 | −2.12 |
| ID 000473 | | | | | | | | |
| Hsa-miR-483-5p | 0.014* | 0.02* | 0.71 | 6.24 ± 1.36 | 4.18 ± 1.28 | 4.18 ± 1.90 | −4.17 | −4.18 |
| ID 002338 | | | | | | | | |
| Hsa-miR-342-3p | 0.002** | 0.008* * | 0.44 | 2.72 ± 0.43 | 1.85 ± 0.66 | 1.67 ± 0.66 | −1.83 | −2.07 |
| ID 002260 | | | | | | | | |

No differently expressed microRNAs were found in spinal onset ALS patients with bulbar impairment.

Validation phase-identification of miR-150-5p and miR-483-5p circulating as biomarkers of bulbar onset ALS.

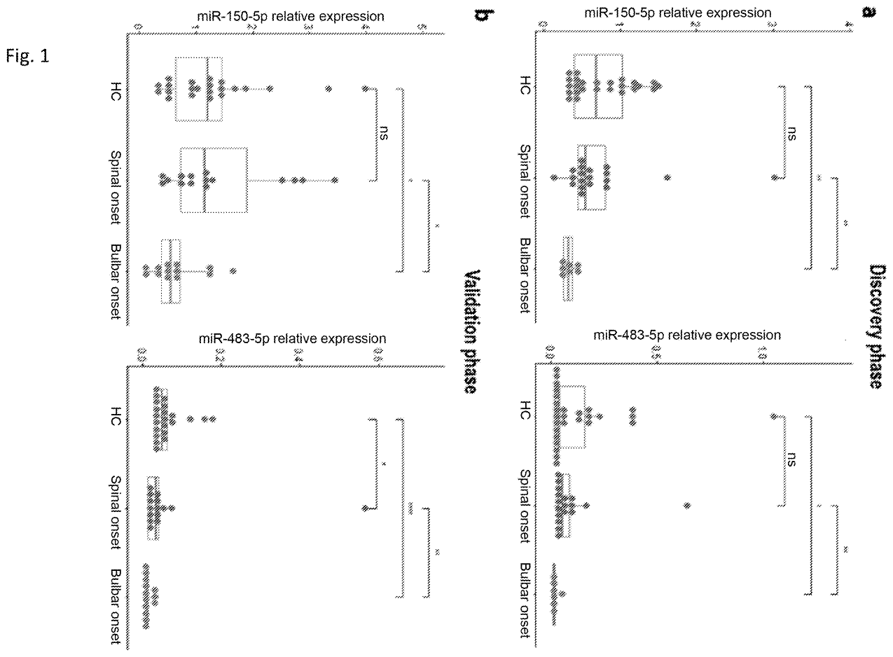

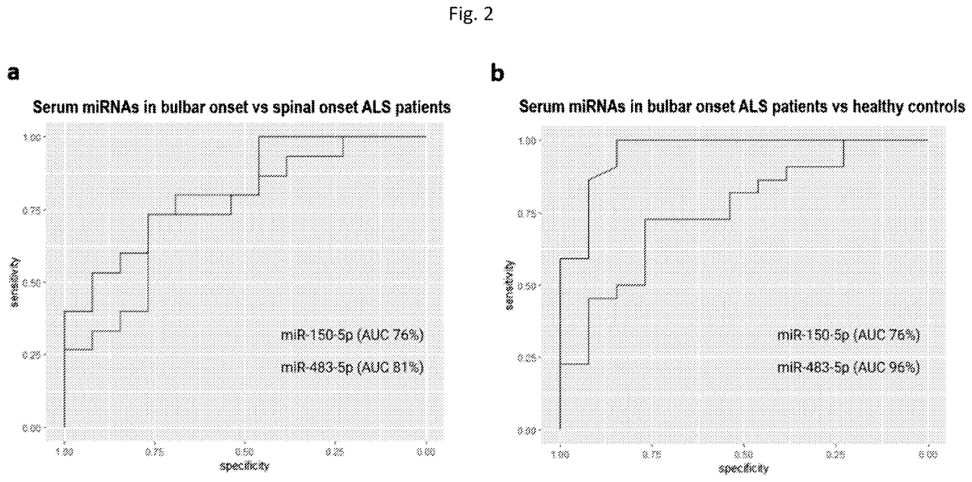

To validate the data obtained in the exploration phase, the four microRNAs, selected in the exploration phase, were analyzed by Real time PCR in the serum of another 28 patients with ALS and 22 HC. The results showed a significant down-regulation of the expression levels of miR-150-5p and miR-483-5p in patients with bulbar onset with respect to patients with spinal onset and HC (FIG. 1), in line with what was observed during the exploration phase. The Receiver Operating Characteristic (ROC) curve analysis showed that the expression levels of the circulating miRNAs, miR-150-5p and miR483-3p, can discriminate the bulbar onset from the spinal phenotype with high sensitivity and specificity (miR-150-5p AUC=76% and miR-463-5p AUC=81%) and HC (miR-150-5p AUC=76% and miR-463-5p AUC=96%) (FIG. 2). To test whether the expression levels of miR-150-5p and miR-483-5p were correlated with each other, Spearman's correlation analysis was performed, which did not find a significant correlation between serum miRNA levels (r=0.26), suggesting distinct pathophysiological roles of the two miRNAs at different regulation levels.

### Proteomic Analysis

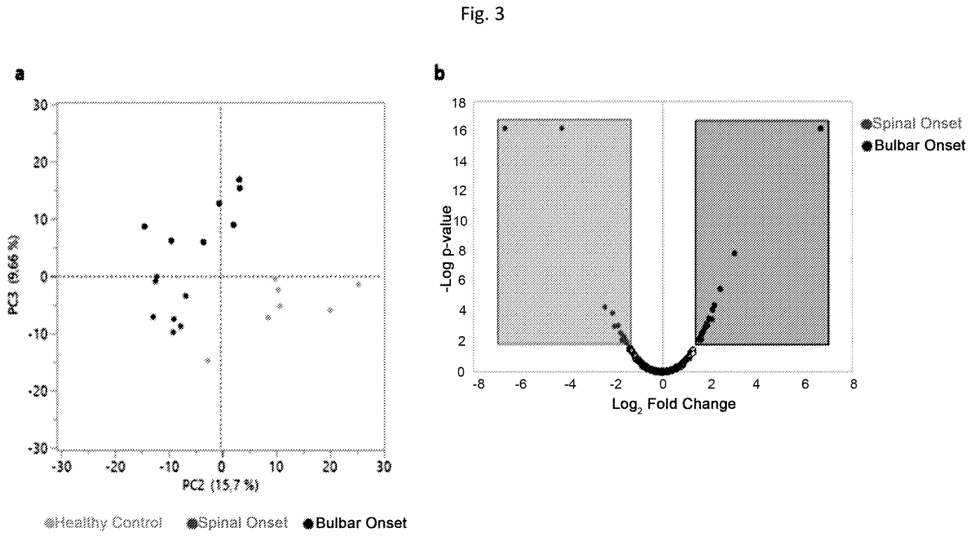

Along with the microRNA expression analysis, a proteomic analysis was carried out on the extracellular vesicles (EV) obtained from the serum of 7 patients with bulbar onset ALS, 7 patients with spinal onset ALS, and 6 HC by means of a platform based on the coupling of liquid nanochromatography and high-resolution tandem mass spectrometry (nLC-hrMS/MS). A total of 1, 175 proteins with at least one unique peptide were identified and unlabeled compared across the three conditions examined, based on the peak intensity of the unique peptide precursor ions. The distribution of proteins and the abundance thereof among the groups were evaluated with Venn diagrams and the box-plot, respectively, resulting in a high percentage of shared proteins (82%) and a very similar distribution of the normalized abundances across the sample groups. The volcano plot and the PCA (FIG. 3) identified three protein clusters which indicate a distinct proteomic profile of bulbar onset ALS patients with respect to spinal onset patients and healthy controls.

To study the differentially expressed proteins, the three groups were compared in pairs (patients with bulbar onset vs. HC; patients with spinal onset vs. HC; patients with bulbar vs. spinal onset) considering the grouped abundance values and applying confidence thresholds of 1.5 for Log 2 Fold Change (FC) and 0.05 for the corrected p-value. A total of 295 proteins were identified which exceeded the thresholds imposed in at least one comparison: 98 up-regulated and 96 down-regulated proteins were identified among patients with bulbar onset and HC; 103 up-regulated and 100 down-regulated proteins among patients with spinal onset and HC; 80 up-regulated and 89 down-regulated proteins among patients with bulbar and spinal onset. The proteomic analyses showed a distinct protein profile, with extreme FC values and p-value for the bulbar onset condition, strongly suggesting that the selected molecular factors could influence this phenotype. A functional network, based on the STRING annotation terms of the biological process, was built to display the connections of protein levels and the biological pathways thereof. The possible interactions between proteins are highlighted and suggest proteins differentially expressed in patients with bulbar ALS with respect to those with spinal onset involved in genetic processing, immune response, endomembrane system, adhesion motility, proliferation, protein metabolism, muscle, stress response, energy metabolism. The network shows the distinct expression of the protein profile for the phenotype with bulbar onset.

Integration of microRNA and Proteomic Profile in the Serum of Patients Affected by Bulbar Onset ALS

An integration analysis between the two candidate miRNAs, miR-150-5p and miR-483-5p, and the differentially selected proteins expressed in the patients affected by bulbar onset versus spinal onset ALS and HC, was carried out to discover distinct molecular signatures. Spearman's correlation analysis was applied to each miRNA and to the abundance of the selected proteins to identify the relationships therebetween. Since miRNAs and RNA-binding proteins (RBPs) have an ever-increasing connection based on mutual regulation, positive and negative correlations were analyzed using coefficients (r) ≥0.5 or ≤−0.5 with p-value<0.05 as a measure of direct and inverse correlations. Three positive and ten negative correlations were identified between miR-150-5p and the selected proteins which are exclusively associated with the bulbar onset phenotype. It should be noted that a positive correlation was found between miR-150-5p and DDP9 protein and negative correlations between and reticulum membrane protein complex (EMC3), Grainyhead Like Transcription Factor 2 (GRHL2) and Dolichyl-Diphosphooligosaccharide-Protein Glycosyltransferase Non-Catalytic Subunit Proteins (DDSOT) whose genes are predicted targets of miR-150-5p. Moreover, thirty-one negative correlations and twenty-five positive correlations were found between miR-483-5p and the selected proteins expressed exclusively in the bulbar onset phenotype. Interestingly, the predicted miRNA target, DENN Domain Containing 6A (DENND6A), positively correlated with miR-483-5p. The biological processes and functions associated with selected molecules in are patients affected by bulbar onset ALS immune response, endomembrane system, protein metabolism, energy metabolism, synapse, ion homeostasis, adhesion-motility, muscle, stress response, apoptosis, genetic information processing, and proliferation. The molecular integration data showed possible mechanisms mediated by microRNAs at distinct regulation levels, confirming the separate and unrelated action of the two selected microRNAs.

A functional network was constructed to display the connections of two microRNAs with positively/negatively correlated proteins and protein linkages with annotated biological processes based on STRING annotation. Biological processes are involved in immune response, endomembrane system, protein metabolism, energy metabolism, synapse, ion homeostasis, adhesion-motility, muscle, stress response, apoptosis, genetic information processing and proliferation.

Analysis of miR-106b-5p Expression Levels in Bulbar Onset Patients Treated with Guanabenz.

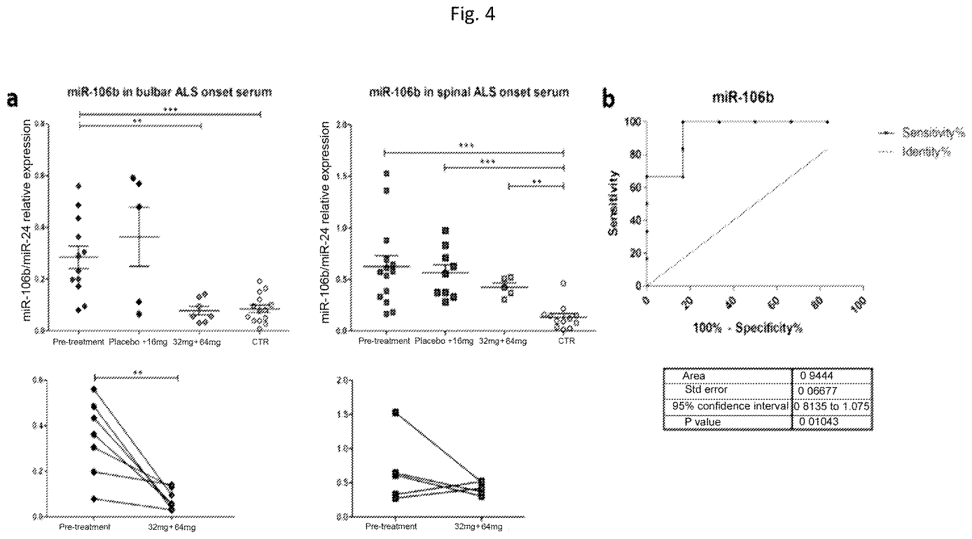

An analysis of miR-106b-5p expression levels in the serum of 12 bulbar onset patients, 5 placebo/16 mg and 7 32 mg/64 mg guanabenz-treated patients, and 14 spinal patients, including 10 placebo/16 mg and 5 32 mg/64 mg guanabenz-treated patients, was performed (FIG. 4). The molecular analyses show a normalization of miR-106b-5p expression levels in the serum of bulbar onset ALS patients treated with 32 mg/64 mg guanabenz with respect to placebo/16 mg drug-treated patients. In contrast, miR-106b-5p expression levels remained significantly higher in patients with spinal onset and in all treatment groups.

### Methods

### Healthy Patients and Controls

The study included 54 deeply phenotyped ALS patients who met the diagnostic criteria for ALS and 50 age- and sex-matched HCs. The patients were grouped as bulbar onset or spinal onset based on published validated criteria. Serum samples obtained from 26 ALS and 28 HC were used for miRNA analyses (discovery phase), while serum samples from a further 28 ALS patients and 22 HC were used for miRNA validation experiments (validation phase). For the proteomic analysis, 7 patients with bulbar onset and 7 spinal onset and 6 HC matched for age and sex were selected, all previously analyzed for miRNA profiling. ALS and HC showed no significant sex-related differences in both the discovery (p-value=0.67) and validation (p-value=0.85). Age was not significantly different comparing ALS against HC in both discovery (p-value t-test=0.74) and validation (p-value t-test=0.62). The study was conducted in accordance with the ethical standards of the Declaration of Helsinki. The investigation and use of patient data for research purposes was approved by the Ethics Committee of the Fondazione IRCCS Istituto Neurologico Carlo Besta in accordance with the Declaration of the World Medical Association.

### Genetic Screening

All patients underwent next-generation sequencing (NGS) of ALS-associated genes by deep amplicon sequencing using the Sure Select QXT kit (Agilent) for SOD1, FUS, TARDBP, VCP, OPTN, SQSTM1, TUBA4A, PFN1, UBQLN2 and Amplidex PCR repetition. Kit PCR/CE C90RF72 (Asuragen Inc. Austin TX) for detection of C9orf72 expansion. The DNA was extracted from peripheral blood using a standard procedure.

The variants were filtered with the following criteria: (i) variants in the coding region or in the flanking 20 bp (ii) absent or rare variants with allelic frequency (AF)<1% in the population databases (dbSNP137, ESP6500, 1000 Genomes project, and ExAC). Taking into account the very low prevalence of ALS, genetic variants with frequency>18 in dbSNP or in Exome Variant Server or ExAC were classified as “benign” (Class-1) and excluded from subsequent analyses. Silico prediction of the effect of synonymous or intronic variants on the junction site was carried out using at least two junction prediction tools: NNSplice predictor (//www.fruitfly.org/seq tools/splice.html); ASSP (http://wangcomputing.com/assp/). The best candidate variants were verified using Sanger methods. The genetic variants that passed the filtering process were classified as “pathogenic” (Class 5), “probably pathogenic” (Class 4), “variants of unknown significance” (VUS, Class 3), and “probably benign” (Class −2), according to criteria proposed by the American College of Medical Genetics and Genomics (ACMG).

### MicroRNA Profile and Data Analysis

With the miRNeasy Serum/Plasma kit (Qiagen, Venlo, Netherlands), the total RNA was extracted from the serum obtained from 26 ALS patients and 28 healthy controls. The RNA quality was checked by 2100Nano Bioanalyzer (Agilent Technologies) and RNA was reverse transcribed using Megaplex RT Human Pool A and B primers and MultiScribe Reverse Transcriptase kit. The cDNA, corresponding to 170 ng of total RNA, was combined with TaqMan Universal PCR Master Mix and distributed in each port of the TaqMan Human MicroRNA A and B v2.0 array board, following the manufacturer's instructions. The arrays were run on the Viia 7 Fast Real-Time PCR system (Thermo Fisher Scientific, Waltham, MA, USA). The human array boards A and B contained primers for 754 miRNAs, including 3 positive control miRNAs and 1 negative control. The relative threshold method (Crt) was applied. Only the miRNAs with good amplification quality (amplification score>1 and Cq confidence>0.8) were included in the analysis. The differential expression was quantified as relative quantification by the 2-ΔΔct method with miR-24 as endogenous control 42 for normalization and spinal SLA or healthy control samples used as reference groups. ΔΔCq was calculated as average ΔCq (miRNA of interest in the bulbar onset ALS group)−average ΔCq (miRNA of interest in the reference group). The fold change in expression was calculated as 2−(ΔΔCq). For a reduction in expression in the interest group with respect to the reference group it was transformed as the negative inverse of 2−(ΔΔCq) to provide the reduction in fold change in the expression.

Validation of miRNA by Real-Time PCR

Total RNA was extracted from the serum of 28 ALS and 22 HC patients using the miRNeasy serum/plasma kit (Qiagen). The RNA quality was checked by 2100Nano Bioanalyzer (Agilent Technologies). The RNA was reverse transcribed using the TaqMan MicroRNA Reverse Transcription Kit with specific primers for the selected miRNAs: miR-150-5p, miR-483-5p, miR-885-5p, miR-342-3p and miR-24 as endogenous control. 43 CDNA (corresponding to 100 mg of total RNA) was amplified in duplicate by real-time PCR, using the universal PCR master mix and pre-designed TaqMan MicroRNA assays on the Viia 7 Fast Real-Time PCR system (Thermo Fisher Scientific). All the results were normalized with respect to miR-24 and the relative miRNA expression levels were calculated using the ΔCt method.

### Protein Extraction and Enzymatic Digestion

The proteomic analysis was performed in EV serum extracts from (N=7) patients with bulbar onset ALS, (N=7) patients with spinal onset ALS, and (N=6) healthy controls, previously analyzed for miRNA experiments. The EVs of 250 μL of pre-purified serum were separated using the ExoQuick ULTRA kit (System Biosciences, Palo Alto, CA, USA), according to the manufacturer's protocol. In short, the serum samples were incubated with ExoQuick42 reagent for min at 4° C., followed by 10 min centrifugation at 3000×g. The pellets were collected, resuspended in 500 μL of ExoQuick Ultra swabs and loaded into a pre-cleaned resin column. After mixing, the purified EVs were obtained by centrifugation at 1000×g for 2 minutes and analyzed to determine the protein concentration with the Qubit™ Protein Analysis Kit (Life Technologies Corp., Eugene, OR, USA). For each sample, 50 μg of protein mixture was reduced/alkylated and enzymatically digested using the Easy Pep Mini MS Sample preparation kit (Thermo Fisher Scientific). Following the kit protocol, in less than 3 hours and for each condition examined, the peptides were generated, cleaned to prepare detergent-free samples, and resuspended in 0.1% formic acid (Sigma-Aldrich Inc., St. Louis, MO, USA) for the nLC-hrMS/MS analysis.

### LC-MS/MS Analysis

The peptide mixtures were analyzed using the Eksigent nanoLC-Ultra 2D system (Eksigent, part of AB SCIEX Dublin, CA, USA) combined with the cHiPLC-nanoflex system (Eksigent) in trap-elute mode. In short, for each condition, two technical replicates were performed by injecting 0.8 μg of protein onto the cHiPLC trap (200 μm×500 μm ChromXP C18-CL, 3 μm, 120 Å, Eksigent, part of AB SCIEX Dublin, CA, USA) and running the loading pump in isocratic mode with 0.1% formic acid in water for 10 minutes at a flow rate of 3 μL/min. The automatic switching of the ten-port valve cHiPLC then eluted the trapped mixture onto a nano cHiPLC column (75 μm×15 cm ChromXP C18-CL, 3 μm, 120 Å, Eksigent, part of AB SCIEX Dublin, CA, USA) through a 115-minute Gradient of eluent B (eluent A, 0.1% formic acid in water; eluent B, 0.1% formic acid in acetonitrile) at a flow rate of 300 nL/min. In depth, the gradient was 5-15% B in 3 min, 15-30% B in 90 min, 30-40% B in 6 min, 40-95% B in 6 min and maintained at 95% B for 11 min. The eluted peptides were analyzed directly on an Orbitrap Exploris 120 mass spectrometer (Thermo Fisher Scientific) provided with EASY-Spray ion source (Thermo Fisher Scientific). The easy spray was obtained using an EASY-Spray emitter (Thermo Fisher Scientific) (nanoflow 7 μm ID Transfer Line 20 μm×50 cm) maintained at 1.6 kV, while the ion transfer capillary was maintained at 220° C. Data dependent acquisition (DDA) was carried out, acquiring precursor ions in the m/z range of 375-1, 250 with a resolution (at m/z 200) of 60,000 FWHM (full width at half maximum). The fragmentation of the precursors was carried out with a resolution (at m/z 200) of 15,000 FWHM, using the high-energy collisional dissociation (HCD) method with normalized collision energy (NCE) of 30 eV and a dynamic exclusion of 20 sec. The MS and MS/MS data were acquired in profile and centroid mode, respectively, using positive polarity and active isotope exclusion. The insulation width was set to 2 m/z and the first mass was set to 120 m/z. The mass spectrometer scan functions and solvent gradients for high performance liquid chromatography were controlled by the Xcalibur version 4.4 data system (Thermo Fisher Scientific) and the Eksigent version 4.3 control software (Eksigent, part of AB SCIEX Dublin, CA, USA), respectively.

### Data Management

All the raw data generated were searched in the Homo Sapiens proteome database (75,550 entries) downloaded in March 2021 from Uniprot (www.uniprot.org) using the Sequest HT search engine contained in the Proteome Discoverer software, version 2.5 (Thermo Fisher Scientific). The MSPepSearch node was inserted into the processing workflow before Sequest HT to carry out a first search in the spectral library (NIST Human Orbitrap HCD library, 1127970 spectra, September 2016). The following criteria were used to identify the peptide sequences and related proteins: mass tolerances of ±10 ppm for precursor ions and ±0.02 Da for fragment ions; trypsin as an enzyme with a maximum of two non-cleavage tolerances; carbamidomethylation of cysteines as a fixed modification; methionine oxidation as a variable modification. The percolator node was used with a target-bait strategy to provide a false discovery rate (FDR) at 1% (strict) and 5% (relaxed), based on q-values, considering XCorr>1.2 and maximum deltaCN of 0.05.43 Label free quantification (LFQ) of the proteins was conducted by setting up a non-nested study with biological and technical replicates, using the recalibration of the spectrum file and the Minora algorithm with the peak intensity of the precursor ion of unique peptides for the abundance calculation when 60% of the replication features was reached. A pair-wise approach was set up for the comparison of the normalized protein abundances between the groups of samples examined. Protein grouping and the strict principle of sparing were also applied. The statistical analysis was carried out on the protein quantification results by a background-based T-test and the differential expressed proteins (DEP) between the experimental groups compared were screened if the log 2-fold change threshold≥|1.5| and the p-value≤0.05 was exceeded. The volcano plots, heat maps and PCA were obtained using the predefined functions of Proteome Discoverer 2.5. The clustering analysis was carried out using the Euclidean mean method for the distance and aggregation of all the proteins.

### Protein Network Analysis

Starting with the DEPs, a protein-protein interaction (PPI) network (268 nodes and 3,532 borders) was constructed from the STRING45 database only experimentally and database-defined PPIs with a score>0.15 were considered. The resulting sub-network was viewed and analyzed by Cytoscape v. 3.9.1 and the plugins. 46, 47 thereof. The proteins were grouped into functional modules by supporting the STRING enrichment, using the default settings. The node colors reflected the DEP expression level in each condition based on normalized abundance cluster values (abundance cluster values were normalized in the range 0-100, setting the highest abundance value per protein to 100).

Correlation Analysis and Functional miRNAs-Proteins-Biological Processes Network Construction

Based on miR-150-5p and miR483-5p expression and protein abundance, Spearman's correlation coefficient and P-value were calculated for each miRNA-protein pair. Spearman's correlation coefficient ≥0.5 or ≤−0.5, with p-values<0.05 was considered statistically significant. Cytoscape (v3.9.1) 48 was used to create and display the functional network linking the miRNAs to proteins and related proteins to related functional biological processes.

### Statistical Analysis

The comparison between demographic and clinical continuous variable groups was carried out using the t-test, while the categorical data were compared with the chi-square test. Multiple comparisons of miRNA expression values in the ALS and HC groups with bulbar, spinal onset were carried out according to the Kruskal-Wallis rank sum test with the post-hoc analysis of Dunn's test, both for the discovery and validation phase. The Benjamini-Hochberg (BH) False Discovery Rate (FDR) test was applied as a correction for multiple tests. The differences were considered statistically significant with an FDR p-value less than 0.05. Spearman's correlation coefficient was used to assess the correlation between miRNA expression levels and the clinical features of the subjects affected by ALS. The ROC curves were used to assess the sensitivity and specificity of miR-150-5p and miR483-5p in human serum samples as a biomarker capable of discriminating between patients affected by bulbar and spinal ALS. The statistical analysis was completed using the statistical programming language R, version 3.6 and STATA11 software.

Analysis of miR-106b-5p Expression Levels

The total RNA was extracted from the serum of 12 bulbar onset patients, 5 placebo/16 mg and 7 32 mg/64 mg guanabenz treated patients, and 14 spinal onset patients, including 10 placebo/16 mg and 5 32 mg/64 mg guanabenz treated patients, using the miRNeasy serum/plasma kit (Qiagen). The RNA quality was checked by 2100Nano Bioanalyzer (Agilent Technologies). The RNA was reverse transcribed using the TaqMan MicroRNA Reverse Transcription Kit with primers specific for the selected miRNAs: miR-106b-5p and miR-24 as endogenous control. cDNA (corresponding to 100 ng of total RNA) was amplified in duplicate by real-time PCR, using the universal PCR master mix and pre-designed TaqMan MicroRNA assays on the Viia 7 Fast Real-Time PCR system (Thermo Fisher Scientific). All the results were normalized with respect to miR-24 and the relative miRNA expression levels were calculated using the ΔCt method.

From the above description the advantages offered by the present invention will be immediately clear.

First, an important new advanced diagnostic tool is provided for a form of amyotrophic lateral sclerosis (ALS) with bulbar onset which has received relatively little attention.

The microRNAs described are circulating molecules in the serum of patients and, therefore, can be identified with simple to conduct and non-invasive methods.

By virtue of what is provided by the present invention, it will be possible to develop new therapeutic approaches for the treatment of amyotrophic lateral sclerosis (ALS) with bulbar onset, which is the most devastating form of this neurodegenerative disease.

The method provided by the present invention has proven to be much more advantageous, as it offers greater reliability and validity, by virtue of the wider database analyzed; moreover, the method of the invention has been validated from a biological and technical point of view.

Overall, therefore, the method is very robust and reliable in discriminating the form of ALS with bulbar onset.

Moreover, such an invention allows a more effective and advanced therapeutic monitoring of guanabenz in patients affected by amyotrophic lateral sclerosis (ALS) with bulbar onset by means of the use of the identified response biomarker.

1. Use of specific microRNAs in the diagnosis of amyotrophic lateral sclerosis (ALS) with bulbar onset, where said specific microRNAs are represented by:

| | |
|---|---|
| miR-150-5p | UCUCCCAACCCUUGUACCAGUG |
| miR-483-5p | AAGACGGGAGGAAAGAAGGGAG. |

2. The use of specific microRNAs in the diagnosis of amyotrophic lateral sclerosis (ALS) with bulbar onset according to claim 1, wherein the expression of said microRNAs is down-regulated.

3. The use of specific microRNAs in the diagnosis of amyotrophic lateral sclerosis (ALS) with bulbar onset, wherein said expression is measured in an isolated serum sample of a patient suspected of having said disease.

4. A diagnostic method for the diagnosis of amyotrophic lateral sclerosis (ALS) with bulbar onset in a patient suspected of having this specific form of disease comprising the step of determining the expression of microRNAs represented by:

| | |
|---|---|
| miR-150-5p | UCUCCCAACCCUUGUACCAGUG |
| miR-483-5p | AAGACGGGAGGAAAGAAGGGAG |

in an isolated biological sample of a patient suspected of having said disease.

5. The diagnostic method according to claim 4, wherein said expression is determined in an isolated serum sample of said patient.

6. A microRNA for medical use in the treatment of patients affected by amyotrophic lateral sclerosis (ALS) with bulbar onset with guanabenz or with drugs acting on the endoplasmic reticulum stress and on the cellular response defined as unfolded protein response, wherein said microRNA is represented by:

| | |
|---|---|
| miR-106b-5p | UAAAGUGCUGACAGUGCAGAU. |

7. The microRNA for medical use in the treatment of patients affected by amyotrophic lateral sclerosis (ALS) with bulbar onset according to the preceding-claim 6, wherein said guanabenz is administered in a dose of 32 mg or 64 mg per day.

8. The microRNA for medical use in the treatment of patients affected by amyotrophic lateral sclerosis (ALS) with bulbar onset according to the preceding-claim 6, wherein the expression value of said microRNA is determined.

9. The microRNA for medical use in the treatment of patients affected by amyotrophic lateral sclerosis (ALS) with bulbar onset according to the claim &, wherein said expression value is determined in an isolated serum sample of said patient.

10. Use of microRNAs for developing a therapeutic approach for amyotrophic lateral sclerosis (ALS) with bulbar onset, wherein said microRNAs are represented by:

| | |
|---|---|
| miR-150-5p | UCUCCCAACCCUUGUACCAGUG |
| miR-483-5p | AAGACGGGAGGAAAGAAGGGAG. |

11. The microRNA for medical use in the treatment of patients affected by amyotrophic lateral sclerosis (ALS) with bulbar onset according to claim 7, wherein the expression value of said microRNA is determined.


SEQUENCE LISTING (3 sequences)

SEQ ID NO: 1 - RNA, 22 nt, Homo sapiens
  source 1..22: mol_type = other RNA
tctcccaacccttgtaccagtg

SEQ ID NO: 2 - RNA, 22 nt, Homo sapiens
  source 1..22: mol_type = other RNA
aagacgggaggaaagaagggag

SEQ ID NO: 3 - RNA, 21 nt, Homo sapiens
  source 1..21: mol_type = other RNA
taaagtgctgacagtgcagat